(19) United States
(12) Patent Application Publication
(10) Pub. No.: US 2002/0138046 A1
(43) Pub. Date: Sep. 26, 2002

(54) Adapter for medication cartridges

(76) Inventor: Joel S. Douglas, Los Altos Hills, CA (US)

Correspondence Address: T. Gene Dillahunty, BURNS, DOANE, SWECKER & MATHIS, L.L.P., P.O. Box 1404, Alexandria, VA, 22313-1404, US

(21) Appl. No.: 10/104,222

(22) Filed: Mar. 22, 2002

Related U.S. Application Data
(60) Provisional application No. 60/278,256, filed on Mar. 23, 2001.
(60) Provisional application No. 60/279,014, filed on Mar. 26, 2001.

(51) Int. Cl. A61M 5/00

(52) U.S. Cl. 604/244

(57) ABSTRACT

An adapter assembly for connecting a cartridge of medication (or any other substance) to a catheter and for securely housing the cartridge within a base or housing, comprising: a body having a bore dimensioned to receive a head of a cartridge therein; a retaining mechanism disposed within the bore in the body, the retaining mechanism dimensioned to hold onto the head of the cartridge; and a double ended hollow needle passing through the first body, the needle having a first end projecting into the bore in the first body; or alternatively; a bottom portion having a bore dimensioned to receive a head of a cartridge therein; a top portion coupled to the bottom portion; a needle hub coupled to the top portion; and a double ended hollow needle passing through the needle hub; wherein an outer surface of the cap is dimensioned to be received into a base.

### RELATED APPLICATION

The present application claims the benefit of priority under 35 U.S.C. Section 119 to U.S. provisional patent application Ser. Nos. 60/278,256 filed Mar. 23, 2001 and 60/279,014 filed Mar. 26, 2001.

### TECHNICAL FIELD

The present invention is related to systems for connecting catheters to medication cartridges.

### SUMMARY OF THE INVENTION

In various embodiments, the present invention provides an adapter assembly comprising: a first body having a bore dimensioned to receive a head of an ampule therein; a retaining mechanism disposed within the bore in the first body, the retaining mechanism dimensioned to hold onto the head of the ampule; a double ended hollow needle passing through the first body, the needle having a first end projecting into the bore in the first body; a second body having a bore dimensioned to receive the first body therein; and a retaining mechanism disposed within the bore in the second body, the retaining mechanism dimensioned to hold onto the first body; and wherein an outer surface of the second body is dimensioned to be received into a base.

In various embodiments, the second body further optionally comprises a septum received therein which is penetrated by a second end of the double ended hollow needle when the first body is received into the bore in the second body.

In alternate embodiments, the present invention provides an adapter assembly comprising: a body having a bore dimensioned to receive a head of an ampule therein; a retaining mechanism disposed within the bore in the body, the retaining mechanism dimensioned to hold onto the head of the ampule; and a double ended hollow needle passing through the body, the needle having a first end projecting into the bore in the body, wherein an outer surface of the body is dimensioned to be received into a base.

In further alternate embodiments, the present invention provides an adapter assembly comprising: a bottom portion having a bore dimensioned to receive a head of an ampule therein; a top portion coupled to the bottom portion; a needle hub coupled to the top portion; and a double ended hollow needle passing through the needle hub; wherein an outer surface of the cap is dimensioned to be received into a base.

In its various embodiments, the present invention provides a system for connecting a medication cartridge (or ampule) to a catheter, while securely holding the medication cartridge (or ampule) in a base or housing such as, for example, an insulin pump.

In accordance with the present invention, the flow path from the medication cartridge (or ampule) to the catheter is opened prior to positioning the cartridge (or ampule) in the base or housing. Although not so limited, thus the present invention is particularly well suited for use with cartridges (e.g. standard ampules) of insulin, wherein the ampule of insulin is conveniently stored in the base or housing of an insulin pump.

As mentioned, the flow path from the medication cartridge (or ampule) to the catheter can be opened prior to positioning the cartridge (or ampule) in the base or housing. Thus, an operator can conveniently view the ampule, and ensure that proper fluid flow is occurring, prior to placing the ampule into the base or housing of the pump (where the ampule cannot be seen). Moreover, in accordance with the present invention, an operator is free to remove the ampule from the base or housing of the infusion pump without having to interrupt the flow of medication from the ampule to the catheter. Thus, the operator may conveniently monitor the amount of fluid remaining in the ampule at different times during use.

In contrast, in pre-existing systems, the flow path from the medication cartridge to the catheter is opened only when the cartridge is positioned within the housing of the infusion pump. Therefore, a user is unable to observe fluid flow from the ampule. Moreover, with such systems, the user is unable to observe fluid level within the ampule without having to first interrupt fluid flow. Such fluid flow interruptions are extremely undesirable for many reasons. For example, continuous delivery of medication (especially insulin) is therapeutically desired. Also, interrupting fluid flow by disconnecting system components increases the risk of contamination.

The present invention also provides a method of connecting an ampule to a catheter, comprising: pushing a top end of an ampule into a bore in a body, the body having a retaining mechanism disposed therein such that the top end of the ampule is held within the bore of the body, the body having a double ended hollow needle passing therethrough such that a first end of the double ended hollow needle passes into the ampule, wherein a second end of the double ended needle is in fluid communication with the catheter; and subsequently, fastening the body into a base. Fastening the body into a base may comprises fastening the body directly into a base, or fastening the body to a second body and then fastening the second body to the base.

### BRIEF DESCRIPTION OF THE DRAWINGS

FIG. 1 is a sectional side elevation view of an embodiment of the present invention.

FIG. 2 is a sectional side elevation view of another embodiment of the present invention.

FIG. 3 is a perspective view corresponding to either of the embodiments of the present invention shown in FIG. 1 or 2.

FIG. 4 is a sectional side elevation view of another embodiment of the present invention.

### DETAILED DESCRIPTION OF THE INVENTION

Before the present articles ad methods are disclosed and described, it is to be understood that the terminology used herein is for the purpose of describing particular embodiments only and is not intended to be limiting. It must be noted that, as used in the specification and the appended claims, the singular forms of “a ”, “an” and “the” include plural referents also unless the context clearly dictates otherwise.

In its various embodiments, the present invention provides adapter assemblies for use with medication cartridges, including pre-filled medication cartridges.

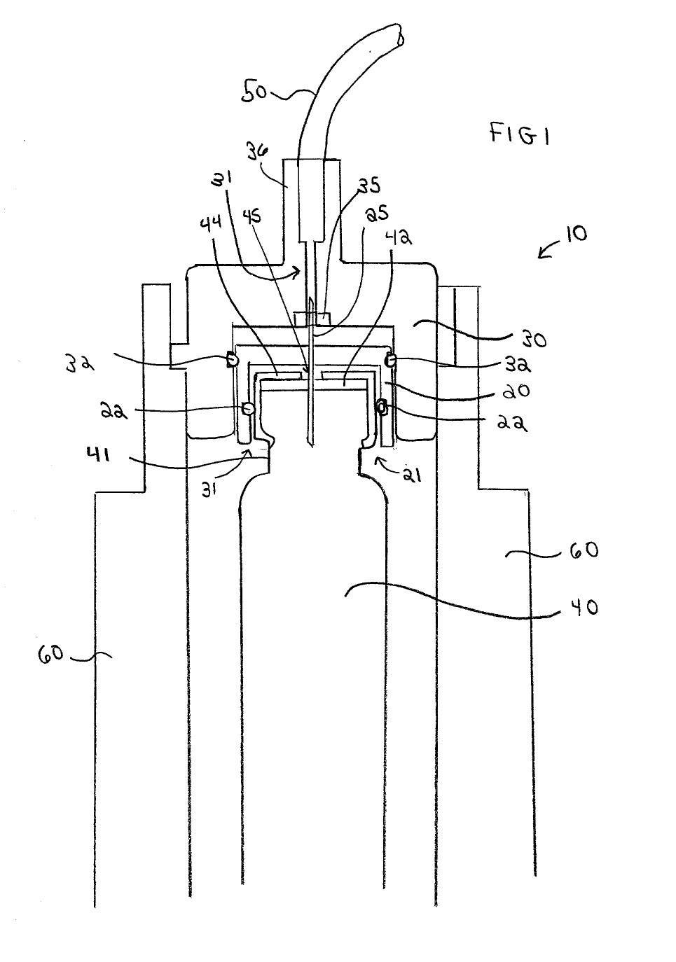

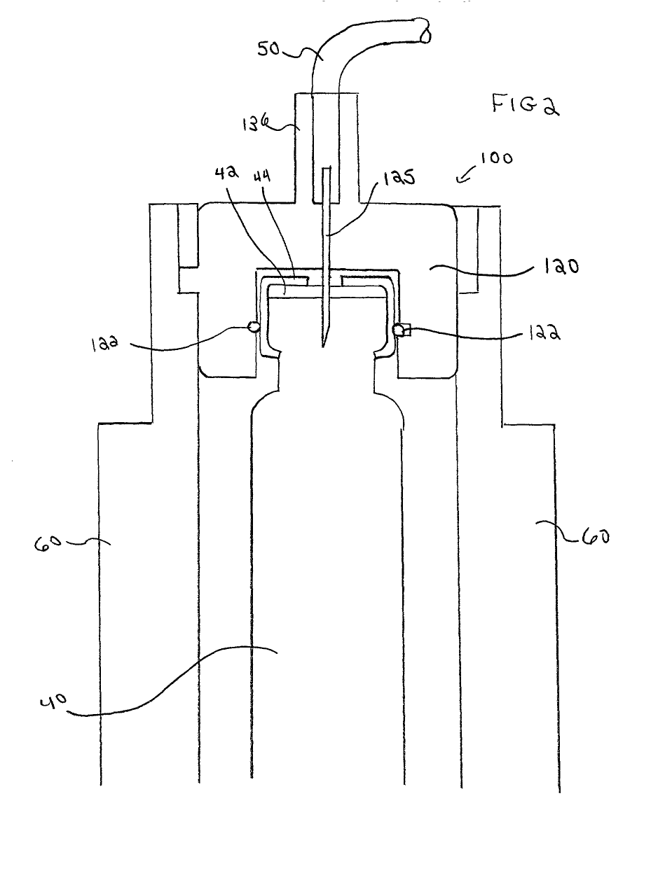

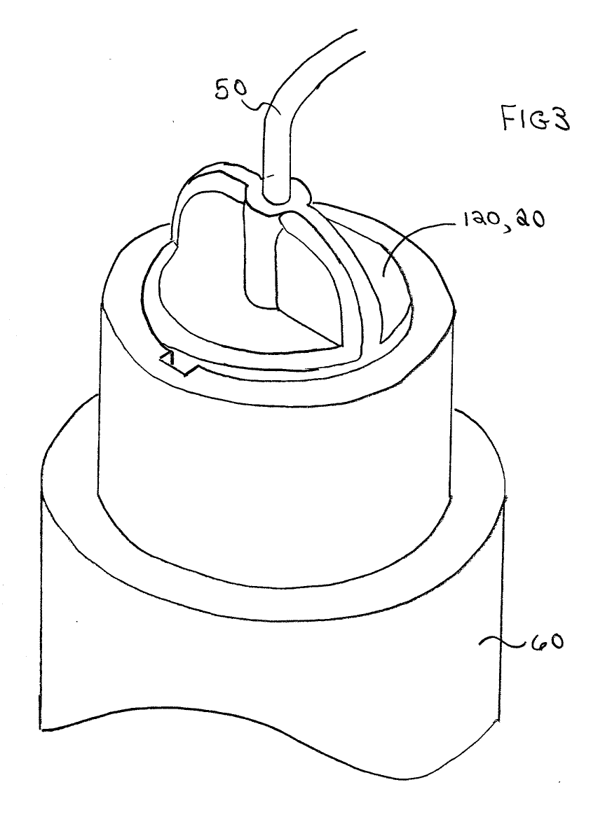

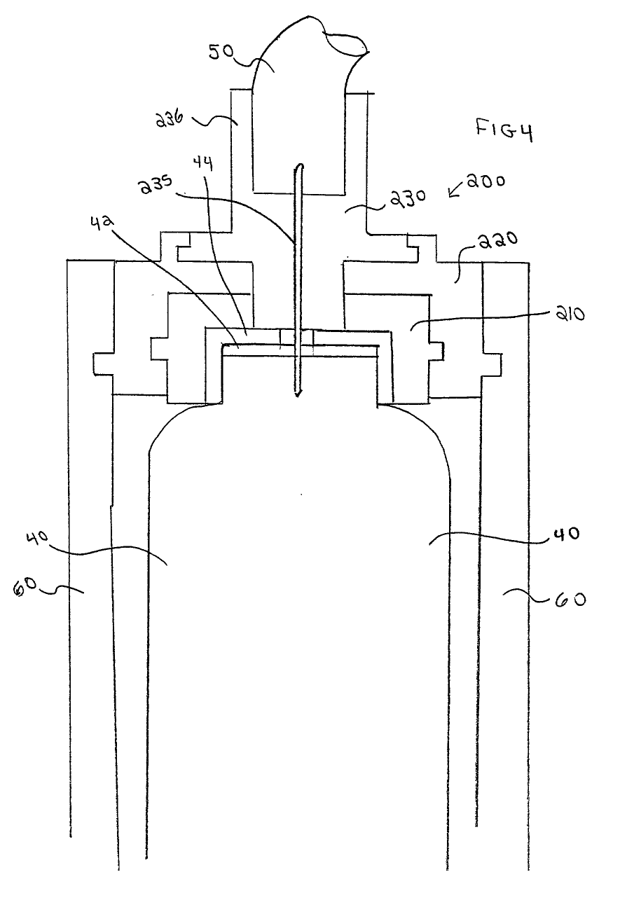

FIGS. 1 and 2 illustrate embodiments of the present invention, showing alternate internal designs. The perspective view of FIG. 3 is an illustration of embodiments of the present invention. Lastly, FIG. 4 illustrates another embodiment of the present invention.

In its various embodiments, the present invention may be used to connect a catheter to an ampule of pre-filled medication such that the flow path from the ampule to the catheter can be opened prior to placing the ampule in a body or housing such as an infusion pump.

An “ampule” is a standard type of cartridge which is typically pre-filled with medication prior to, or during, operation. Such medication may optionally comprise insulin, but is not so limited. Typically, an ampule simply comprises a glass tube having a top end resembling a bottle neck which is sealed by a rubber membrane covering which is in turn held in position by an external flange which wraps partially over the rubber membrane and around the “neck” of the ampule. The external flange has a hole disposed over the center of the rubber membrane, thereby allowing a needle to pass through the rubber membrane and withdraw fluid from inside the ampule. The opposite end of the ampule may be sealed by a piston which is used to expel the contents of the ampule out through the needle passing through the rubber membrane sealing the top of the ampule. However, other systems for sealing the ampule are also contemplated. It is to be understood that the present invention is not limited solely to insulin ampules, or any other sort of ampules. Rather, the present invention is suited for use with any form of medication or substance delivery cartridge. Thus, the present references to an “ampule” are defined to include references to any form of cartridge.

Referring to FIG. 1, an embodiment of the present invention is shown. Adapter assembly 10 is a “two-part” embodiment of the present invention, comprising a first body 20 and a second body 30. The top end of a standard ampule 40 is received into bore 21 in first body 20.

Ampule 40 comprises a tube having a neck 41. As is common with standard ampules, a rubber membrane 42 covers its top end. Rubber membrane 42 is held in position by a metal flange 44 which covers rubber membrane 42 and wraps around neck 41 (with the exception of a small hole 45 at the center of rubber membrane 42).

First body 20 comprises a retaining mechanism, which may comprise an O-ring 22 therein. In accordance with the present invention, O-ring 22 provides a frictional grip on the top end of ampule 40 such that ampule 40 is held in position within first body 22 when it is pushed therein. It is to be understood that O-ring 22 is merely an exemplary restraining mechanism for holding the top end of ampule 40 within first body 20. The present invention is not so limited. Rather, any suitable system (friction-based interference fit, or otherwise) can be used for holding the top end of ampule 40 within first body 20, all keeping within the scope of the present invention.

Second body 30 has a bore 31 which is dimensioned to receive first body 20 therein. Second body 30 comprises a retaining mechanism such as O-ring 32 therein. In accordance with the present invention, O-ring 32 provides a frictional grip between first body 20 and second body 30 such that first body 20 is held within second body 30 when it is pushed therein. It is to be understood that O-ring 32 is an exemplary restraining mechanism for holding first body 20 and second body 30 together. The present invention is not so limited. Rather, any suitable system (friction-based, or otherwise) can be used for holding first body 20 and second body 30 together, all keeping within the scope of the present invention.

First body 20 further comprises a double ended hollow needle 25 passing therethrough. A tubular projection 36 extends from second body 30. A catheter 50 is connected to tubular projection 36. Second body 30 may optionally comprise a septum 35 which covers a narrow portion 34 of bore 31.

Thus, when the top end of ampule 40 is press-fit into first body 20 and first body 20 is press-fit into second body 30, a first end of needle 25 will pass through rubber membrane 42 into ampule 40, and a second end of needle 25 will pass through septum 35, and be disposed in narrow portion 34 of bore 31 in second body 30. At this time, an open fluid flow path with be created from ampule 40 through first body 20 and second body 30 into catheter 50. Thus, an operator can view the contents in ampule 40.

Thereafter, adapter assembly 10 can be positioned within a base 60. In various embodiments, base 60 comprises the housing of an insulin pump. In various embodiments, an outer surface of second body 30 is threaded such that it can conveniently be screw-fit into base 60, (as shown in FIG. 3.). Other systems of fastening second body 30 into base 60 are also contemplated. For example, a detent-fitting system may be used.

Referring next to FIG. 2, a “one-part” embodiment of the present invention is provided. Specifically, adapter assembly 100 comprises a body 120, having a bore 120 into which the top end of a standard ampule 40 is received.

Body 120 comprises a retaining mechanism such as O-ring 122 therein. In accordance with the present invention, O-ring 122 provides a frictional grip on the end of ampule 40 such that ampule 40 is held in position within body 120 when it is pushed therein. It is to be understood that O-ring 122 is an exemplary restraining mechanism for holding the top end of ampule 40 within first body 120. The present invention is not so limited. Rather, any suitable system (friction-based, or otherwise) can be used for holding the top end of ampule 40 within body 120, all keeping within the scope of the present invention.

Body 120 further comprises a double ended hollow needle 125 passing therethrough. A tubular projection 136 extends from body 120. A catheter 50 is connected to tubular projection 136.

Thus, when the top end of ampule 40 is press-fit into body 120, a first end of needle 125 will pass through rubber membrane 42 and into ampule 40, with the second end of needle 125 disposed in tubular projection 136. At this time, an open flow path with be created from ampule 40 through body 120 into catheter 50.

Thereafter, adapter assembly 100 can be positioned within a base 60. In various embodiments, base 60 comprises the housing of an insulin pump. In various embodiments, an outer surface of body 120 is threaded such that it can conveniently be screw-fit into base 60, (as shown in FIG. 3.) Other systems of fastening body 120 into base 60 are also contemplated. For example, a detent-fitting system may be used.

Referring next to FIG. 4, a third “three-part” embodiment of the present invention is shown. Specifically, adapter assembly 200 comprises a bottom portion 210, a top portion 220 and a needle hub 230 which are assembled together. Needle hub 230 further comprises a double ended needle 235 passing therethrough.

Bottom portion 210 may have an outer surface which is threaded, and top portion 220 may have an inner surface which is threaded such that top portion 220 may be screw-fit onto the top of bottom portion 210. Similarly, in various embodiments, an outer surface of top portion 220 is threaded such that it can conveniently be screw-fit into base 60. Other systems of fastening top portion 220 and bottom portion 210 together are also contemplated. For example, a detent-fitting system may be used.

Top portion 220 has an optional lip 224 and needle hub 320 has an optional lip 324 which mate together such that needle hub 320 and top portion 220 can be fastened together. In various embodiments, needle hub 320 is made from a soft, flexible material such that it can be snap-fit under lip 224 and onto the top of top portion 220.

When ampule 40 is received into bottom portion 210, and bottom portion 210 is connected to top portion 220, and top portion 220 is connected to needle hub 230, and a catheter 50 is connected to the hollow tubular extension 326, an open flow path from ampule 40 into catheter 50 is provided.

Thereafter, adapter assembly 200 can be positioned within a base 60. In various embodiments, base 60 comprises the housing of an insulin pump. In various embodiments, an outer surface of top portion 220 is threaded such that it can conveniently be screw-fit into base 60. Other systems of fastening top portion 220 into base 60 are also contemplated. For example, a detent-fitting system may be used.

What is claimed is:

1. An adapter assembly comprising:
a first body having a bore dimensioned to receive a head of an ampule therein;
a retaining mechanism disposed within the bore in the first body, the retaining mechanism dimensioned to hold onto the head of the ampule;
a double ended hollow needle passing through the first body, the needle having a first end projecting into the bore in the first body;
a second body having a bore dimensioned to receive the first body therein; and
a retaining mechanism disposed within the bore in the second body, the retaining mechanism dimensioned to hold onto the first body; and
wherein an outer surface of the second body is dimensioned to be received into a base.

2. The adapter assembly of claim 1, wherein the second body further comprises a septum received therein which is penetrated by a second end of the double ended hollow needle when the first body is received into the bore in the second body.

3. The adapter assembly of claim 1, wherein the retaining mechanism disposed within the bore in the first body comprises:
an O-ring seal.

4. The adapter assembly of claim 1, wherein the retaining mechanism disposed within the bore in the second body comprises:
an O-ring seal.

5. The adapter assembly of claim 1, further comprising:
a hollow tubular projection extending from the adapter, wherein a second end of the double ended hollow needle projects into the hollow tubular projection when the first body is received into the bore in the second body.

6. The adapter assembly of claim 1, wherein the outer surface of the second body is threaded.

7. The adapter assembly of claim 1, wherein the base comprises an infusion pump.

8. An adapter assembly comprising:
a body having a bore dimensioned to receive a head of an ampule therein;
at least one retaining mechanism disposed within the bore in the body, the retaining mechanism dimensioned to hold onto the head of the ampule; and
a double ended hollow needle passing through the body, the needle having a first end projecting into the bore in the body, wherein an outer surface of the body is dimensioned to be received into a base.

9. The adapter assembly of claim 8, wherein the retaining mechanism disposed within the bore in the body comprises an O-ring seal.

10. The adapter assembly of claim 8, further comprising:
a hollow tubular projection extending from the adapter, wherein a second end of the double ended hollow needle projects into the hollow tubular projection.

11. The adapter assembly of claim 8, wherein the outer portion of the body is threaded.

12. The adapter assembly of claim 8, wherein the base comprises an infusion pump.

13. An adapter assembly comprising:
a bottom portion having a bore dimensioned to receive a head of an ampule therein;
a top portion coupled to the bottom portion;
a needle hub coupled to the top portion; and
a double ended hollow needle passing through the needle hub;
wherein an outer surface of the cap is dimensioned to be received into a base.

14. The adapter assembly of claim 13, wherein an outer surface of the top portion is threaded to be fit into the base.

15. The adapter assembly of claim 13, wherein an outer surface of the bottom portion is dimensioned to be received into the top portion.

16. The adapter assembly of claim 13, wherein a first end of the double ended hollow needle penetrates into the ampule when the ampule is received into the bottom portion and the bottom portion, top portion and needle hub are assembled together.

17. The adapter assembly of claim 13, wherein the needle hub comprises a hollow tubular projection extending therefrom, wherein a second end of the double ended hollow needle projects into the hollow tubular projection.

18. The adapter assembly of claim 13, wherein the base comprises an infusion pump.

19. A method of connecting an ampule to a catheter, comprising:
pushing a top end of an ampule into a bore in a body, the body having a retaining mechanism disposed therein such that the top end of the ampule is held within the bore of the body, the body having a double ended hollow needle passing therethrough such that a first end of the double ended hollow needle passes into the ampule, wherein a second end of the double ended needle is in fluid communication with the catheter; and
subsequently, fastening the body into a base.

20. The method of claim 19, wherein fastening the body into a base comprises:
fastening the body to a second body and then fastening the second body to the base.

21. A kit comprising:
an adapter assembly according to claims 1, 8 or 13; and
instructions for use setting forth the method of claim 19.
